(19) United States
(12) Patent Application Publication
(10) Pub. No.: US 2008/0206237 A1
(43) Pub. Date: Aug. 28, 2008

(54) Synergistic Combination Of Xolair/Omalizumab/E25 With Immunosuppressive Agent

(76) Inventors: Charles Edward Owen, West Sussex (GB); Howard George Fox, West Sussex (GB); Christoph Walker, West Sussex (GB)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/814,871

(22) PCT Filed: Feb. 2, 2006
(86) PCT No.: PCT/EP06/00911

§ 371 Date: Aug. 24, 2007

(30) Foreign Application Priority Data
Feb. 4, 2005 (GB) 0502358.5

(51) Int. Cl. A61K 39/395; A61P 37/08

(52) U.S. Cl. 424/131.1

(57) ABSTRACT

The present invention provides a pharmaceutical composition comprising an Anti-IgE Antibody and at least one further immunosuppressive agent, in which the active ingredients are present in each case in free form or in the form of a pharmaceutically acceptable salt and optionally at least one pharmaceutically acceptable carrier; for simultaneous, separate or sequential use.

### BACKGROUND OF THE INVENTION

The generally accepted aims in the treatment of allergic disease, are to provide relief from symptoms, improvement of the quality of life and prevention of both acute and chronic complications. Treatment of allergic disease varies with the severity and type of the symptoms. Short-term goals include relieving immediate symptoms, while long-term goals also include avoiding future allergic reactions. In order to achieve the therapeutical goals, it is often necessary to give medication to the patient having allergic disease. For example, corticosteroids such as dexamethasone or prednisone reduce the immune response and may be prescribed to reduce symptoms in allergic disease, antihistamines such as diphenhydramine may provide good relief of mild to moderate symptoms and epinephrine may be used to reduce swelling of the airways and other life-threatening symptoms of allergic disease. Generally, avoidance of the allergen is important for long-term treatment, particularly with allergic reaction to foods or medications. Also, desensitization (immunotherapy) is occasionally recommended if the allergen cannot be avoided. Desensitization includes regular injections of the allergen, given in increasing doses.

For example, in allergic asthma the treatment is aimed at controlling symptoms through medication. A variety of medications for treatment of allergic asthma are available. These medications include antiallergic compounds of various chemical and therapeutical classes, such as, for example, anti-inflammatory substances, leukotriene inhibitors, bronchodilators, cromolyn sodium and amino- or theophylline. Patients with mild asthma, i.e. having infrequent attacks, may use bronchodilators as needed while those with significant asthma, e.g. symptoms occurring more than twice per week, should be treated with anti-inflammatory medications, preferably inhaled corticosteroids, and with inhaled bronchodilators in addition. Acute severe asthma requires a medical evaluation and may require hospitalization, oxygen, and intravenous medications.

However, there generally remains a need to improve the presently available medication in order to better control the symptoms and to ameliorate the underlying disease processes in order to meet the therapeutic challenge of controlling allergic disease.

### SUMMARY OF THE INVENTION

The present invention provides a pharmaceutical composition comprising as active ingredients an Anti-IgE Antibody and at least one agent suppressing immune response. The active ingredients are present in each case in free form or in the form of a pharmaceutically acceptable salt and optionally at least one pharmaceutically acceptable carrier; for simultaneous, separate or sequential use.

Also provided is a method for the prevention, delay of progression or treatment of allergic disease comprising administering to a warm-blooded animal a therapeutically effective amount of the composition of the invention.

In another aspect of the invention there is provided the use of the composition of the invention in medicine.

Furthermore, the use of a composition according to the invention for the manufacture of a medicament for the treatment of a warm-blooded animal having allergic disease is provided.

In another aspect, there is provided a kit comprising as active agent a composition according to this invention together with instructions for simultaneous, separate or sequential use thereof in the prevention, delay of progression or treatment of allergic disease.

### DETAILED DESCRIPTION OF THE INVENTION

The present invention relates to a combination, such as a combined preparation or pharmaceutical composition, respectively, which comprises an Anti-IgE Antibody and at least one immunosuppressive agent, in which the active ingredients are present in each case in free form or in the form of a pharmaceutically acceptable salt and optionally at least one pharmaceutically acceptable carrier; for simultaneous, separate or sequential use, especially in the prevention, delay of progression or treatment of allergic disease, especially allergic asthma, seasonal allergic rhinitis, perennial allergic rhinitis, atopic dermatitis, chronic rhinosinusitis, COPD, food allergy such as e.g. peanut allergy and diseases and conditions associated with allergic disease and diseases and conditions associated with allergic disease.

The anti-IgE antibody and at least one immunosuppressive agent can be dosed independently or by use of different fixed combinations with distinguished amounts of the components. The parts of the kit of parts can then e.g. be administered chronologically staggered, that is at different time points and with equal or different time intervals for any part of the kit of parts. Preferably, the time intervals are chosen such that the effect on the treated disease or condition in the combined use of the parts is larger than the effect which would be obtained by use of only any one of the components. Preferably, there is at least one beneficial effect, e.g. a mutual enhancing of the effect of the active ingredients, additional advantageous effects, less side-effects, a combined therapeutical effect in a non-effective dosage of one or each of the active ingredients, and especially a synergism, e.g. a more than additive effect, between an anti-IgE antibody, respectively, and the at least one further compound as mentioned above.

An “Anti-IgE antibody” within the meaning of the invention may be any antibody directed against an IgE antibody, in particular an antibody directed against the Fc portion of an IgE antibody. Preferably the anti-IgE antibody is a humanized murine antibody or a fully human antibody. Preferably the anti-IgE antibody is a non-anaphylactogenic anti-IgE antibody. Thus, preferably, the IgE antibodies of the instant invention do not result in histamine release from mast cells or basophils.

Preferred anti-IgE antibodies of the invention are the antibodies named Omalizumab (E25), E26, CGP56901, CGP51901, or their fragments and derivatives, as further defined hereinbelow. Most preferably the anti-IgE antibody is Omalizumab, which is also named “E25” or “Xolair®.” Another particularly preferred anti-IgE antibody is named “E26” as further defined hereinbelow.

Generally, anti-IgE antibodies are described in the prior art, and in greater detail in the International applications WO 93/04173 and WO 99/01556. In each case where citations of patent applications are given, the subject matter relating to the compounds is hereby incorporated into the present application by reference. For example, WO 99/01556 specifically describes Omalizumab, in FIG. 12, and in the sequences ID-No. 13-14. Antibody molecules comprising a E26 sequence are described in WO 99/01556 and are selected from the group of F(ab) fragment (Sequence ID Nos. 19-20), sFv fragment (Sequence ID No. 22) and F(ab)′₂ fragment (Sequence Nos. 24-25), in accordance to FIGS. 12-15. Within this invention, the terms E25 and E26 shall be construed accordingly.

Also included in the present invention are the antibodies as specifically described in US patents U.S. Pat. No. 6,066,718; U.S. Pat. No. 6,072,035 and U.S. Pat. No. 5,958,708.

U.S. Pat. No. 5,449,760 generally describes anti-IgE antibodies that bind soluble IgE but not IgE on the surface of B cells or basophils. Antibodies such as these bind to soluble IgE and inhibit IgE activity by, for example, blocking the IgE receptor binding site, by blocking the antigen binding site and/or by simply removing the IgE from circulation. Additional anti-IgE antibodies and IgE-binding fragments derived from the anti-IgE antibodies are described in U.S. Pat. No. 5,656,273. U.S. Pat. No. 5,543,144 describes anti-IgE antibodies that bind soluble IgE and membrane-bound IgE on IgE-expressing B cells but not to IgE bound to basophils. The term “immunosuppressant” or “immunosuppressive agent” within the context of the present application refers to a compound useful to prevent the rejection of transplanted organs (allograft rejection). The “immunosuppressive agent” of the invention may be selected from the group consisting of (1) inhibitor of T-helper cell activation, (2) calcinneurin inhibitor, (3) IL-2 antagonist, (4) inhibitor of T-Lymphocyte proliferation, (5) inhibitor of T-Lymphocyte migration.

Currently known immunosuppressants include without limitation a calcineurin inhibitor, e.g. a cyclosporin or an ascomycin, e.g. Cyclosporin A (NEORAL®), ISAtx-247, FK506 (tacrolimus), FK778, ABT-281 or ASM981, a mTOR inhibitor, e.g. rapamycin or a derivative thereof, e.g. Sirolimus (RAPAMUNE®), Everolimus (Certican®), CCI779, ABT578, biolimus-7, biolimus-9, a rapalog, e.g. AP23573, azathioprine, campath 1H, a S1P receptor modulator, e.g. FTY720 or an analogue thereof, an anti IL-8 antibody, mycophenolic acid or a salt thereof, e.g. sodium salt, or a prodrug thereof, e.g. Mycophenolate Mofetil (CELLCEPT®), OKT3 (ORTHOCLONE OKT3®), Prednisone, ATGAM®, THYMOGLOBULIN®, Brequinar Sodium, OKT4, T10B9.A-3A, 33B3.1, 15-deoxyspergualin, tresperimus, Leflunomide ARAVA®, CTLA1-Ig, anti-CD25, anti-IL2R, Basiliximab (SIMULECT®), Daclizumab (ZENAPAX®), mizorbine, methotrexate, dexamethasone, ISAtx-247, SDZ ASM 981 (pimecrolimus, Elidel®), CTLA4Ig (Abatacept), LEA29Y, LFA3Ig, hu5C8, etanercept (sold as Enbrel® by Immunex), adalimumab (Humira®), infliximab (Remicade®), an anti-LFA-1 antibody, natalizumab (Antegren®), UO126, B7RP-1-fc, hul 124, BTI-322, allotrap-HLA-B270, Enlimomab, ABX-CBL, antithymocyte immunoglobulin, Medi-500, Medi-507, Alefacept and efalizumab.

In one aspect the present invention provides a pharmaceutical composition comprising Xolair® and at least one immunosuppressive agent. The immunosuppressive agent is preferably selected from the group consisting of a a calcineurin inhibitor, e.g. a cyclosporin or an ascomycin, e.g. Cyclosporin A (NEORAL®), ISAtx-247, FK506 (tacrolimus), FK778, ABT-281 or ASM981, a mTOR inhibitor, e.g. rapamycin or a derivative thereof, e.g. Sirolimus (RAPAMUNE®), Everolimus (Certican®), CCI779, ABT578, biolimus-7, biolimus-9, a rapalog, e.g. AP23573, azathioprine, campath 1H, a S1P receptor modulator, e.g. FTY720 or an analogue thereof, an anti IL-8 antibody, mycophenolic acid or a salt thereof, e.g. sodium salt, or a prodrug thereof, e.g. Mycophenolate Mofetil (CELLCEPT®), OKT3 (ORTHOCLONE OKT3®), Prednisone, ATGAMO, THYMOGLOBULIN®, Brequinar Sodium, OKT4, T10B9.A-3A, 33B3.1, 15-deoxyspergualin, tresperimus, Leflunomide ARAVA®, CTLA1-Ig, anti-CD25, anti-IL2R, Basiliximab (SIMULECT®), Daclizumab (ZENAPAX®), mizorbine, methotrexate, dexamethasone, ISAtx-247, SDZ ASM 981 (pimecrolimus, Elidel®), CTLA4Ig (Abatacept), LEA29Y, LFA31g, hu5C8, etanercept (sold as Enbrel® by Immunex), adalimumab (Humira®), infliximab (Remicade®), an anti-LFA-1 antibody, natalizumab (Antegren®), UO126, B7RP-1-fc, hul 124, BTI-322, allotrap-HLA-B270, Enlimomab, ABX-CBL, antithymocyte immunoglobulin, , Medi-500, Medi-507, Alefacept and efalizumab.

In a preferred embodiment, the present invention provides a pharmaceutical composition comprising Xolair® and at least one immunosuppressant antibody, preferably selected from the group consisting of an anti-IL2R antibody, an anti-CD25 antibody, basiliximab, daclizumab, an anti IL-8 antibody, adalimumab, infliximab, hu5C8, OKT3, OKT4, anti-TACac, T10B9.A-3A, 33B3.1, hul 124, BTI-322, allotrap-HLA-B270, Enlimomab, ABX-CBL, antithymocyte immunoglobulin, Medi-500, Medi-507, Alefacept and efalizumab. In a particularly preferred embodiment, the immunosuppressant is anti-IL2R antibody, e.g. basiliximab or daclizumab.

In another preferred embodiment, the immunosuppressant is a calcineurinhibitor, preferably selected from the group consisting of cyclosporin A, FK506 or pimecrolimus.

In yet another preferred embodiment, the immunosuppressant inhibits T-cell activation by blocking the IL-2 receptor such as e.g. an anti-IL2R antibody, an anti-CD25 antibody, basiliximab or daclizumab. T-cell activation and cytokine secretion appear to play central roles in the generation and potentiation of airway inflammation in chronic asthma. Patients with severe steroid-resistant asthma have been found to have significantly higher proportions of IL-2 receptor-positive activated T cells and elevated IL-2 levels in cultures of peripheral blood leukocytes. Targeting IL-2 receptors expressed on activated T cells inhibits the cascade of immune events that leads to airway inflammation and destruction in asthma. In accordance with the present invention, it has now been found that a combination of an anti- IgE antibody with an IL-2R dependent inhibitor T-cell activation is particularly effective in the treatment or prevention of allergic responses.

Conventional corticosteroid treatment has positive therapeutic benefit primarily by effecting T helper 2 cell responses, but has little impact the IgE pathway. A combination of an effective T cell inhibitor, such as an anti-CD25 antibody, with Xolair has, in accordance with the present invention, added therapeutic benefit. The combination in accordance with the present invention also provides therapeutic benefit by allowing treatment of both adaptive and humoral immune response concomitantly. Furthermore, in accordance with one embodiment of the present invention, immunosuppressive agents are therapeutically beneficial at sub-toxic doses when dosed in combination with Xolair.

In a preferred embodiment, the immunosuppressant inhibits the T-cell activation by at least 10%, 20%, 50%, 80%, 90%, 95% or 99%. Inhibition of T-cell activation can be measured by a suitable assay using e.g. an assay for T-cell activation wherein purified T-cells are stimulated with an activator (e.g. a monoclonal antibody or a cytokine) and proliferation is measured in the presence and in the absence of the T-cell inhibitor (see e.g. Anderson et al., Nature Medicine, 2000, 6: 211-214; Staffler et al., The Journal of Immunology, 2003, 171: 1707-1714).

An IL-2R antagonist is an agent that binds to the IL-2R (Interleukin 2 Receptor) on activated T-lymphocytes and inhibits the activity of the receptor. IR-2R antagonists include agents that bind specifically to the alpha chain (or CD25) of the human IL-2R, such as daclizumab, basiliximab, BT563, and 7G8 (collectively known as anti-CD25 antibodies), or agents that bind to other subunits, such Mig beta-2, which binds to the beta chain of human IL-2R.

In specific embodiments the IL-2R antagonist is an anti-CD25 antibody. In some cases, the anti-CD25 agent is daclizumab (Zenapax®). Daclizumab is an immunosuppressive, humanized IgG1 monoclonal antibody produced by recombinant DNA technology that binds specifically to the alpha subunit (p55 alpha, CD25, or Tac subunit) of the human high-affinity interleukin-2 (IL-2) receptor that is expressed on the surface of activated lymphocytes. Daclizumab is a composite of human (90%) and murine (10%) antibody sequences. The human sequences were derived from the constant domains of human IgG1 and the variable framework regions of the Eu myeloma antibody. The murine sequences were derived from the complementarily-determining regions of a murine anti-Tac antibody. The molecular weight predicted from DNA sequencing is 144 kilodaltons.

In other embodiments the anti-CD25 agent is basiliximab (Simulect®). Simulect is a chimeric (murine/human) monoclonal antibody (IgG 1 k), produced by recombinant DNA technology, that functions as an immunosuppressive agent, specifically binding to and blocking the alpha chain of the IL-2R on the surface of activated T-lyrnphocytes. Basiliximab is a glycoprotein obtained from fermentation of an established mouse myeloma cell line genetically engineered to express plasmids containing the human heavy and light chain constant region genes and mouse heavy and light chain variable region genes encoding the RFT5 antibody that binds selectively to the IL-2R (alpha). Based on the amino acid sequence, the calculated molecular weight of the protein is 144 kilodaltons.

In some examples, the IL-2R antagonist is a combination of anti-CD25 antibodies. For example, daclizumab and basiliximab are administered together as a cocktail, or the agents are alternated in the administration schedule.

The structure of the active agents identified by code nos., generic or trade names may be taken from the actual edition of the standard compendium “The Merck Index” or the “Physician's Desk Reference (PDR)” or from databases, e.g. Patents International (e.g. IMS World Publications). The corresponding content thereof is hereby incorporated by reference. A person skilled in the art is fully enabled to identify the active agents and, based on these references, likewise enabled to manufacture and test the pharmaceutical indications and properties in standard test models, both in vitro and in vivo.

It will be understood that in the discussion of methods, references to the active ingredients are meant to also include the pharmaceutically acceptable salts. If these active ingredients have, for example, at least one basic center, they can form acid addition salts. Corresponding acid addition salts can also be formed having, if desired, an additionally present basic center. The active ingredients having an acid group (for example COOH) can also form salts with bases. The active ingredient or a pharmaceutically acceptable salt thereof may also be used in form of a hydrate or include other solvents used for crystallization.

The combination which comprises an anti-IgE antibody such as, for example, Omalizumab (Xolair®) and at least one immunosuppressive agent, is present in each case in free form or in the form of a pharmaceutically acceptable salt, if at least one salt-forming group is present, will be referred to hereinafter as a COMBINATION OF THE INVENTION. A preferred combination in accordance with the present invention is Omalizumab with at least one immunosuppressive agent selected from the group consisting of Cyclosporin A (NEORAL®), ISAtx-247, FK778, ABT-281 or ASM981, azathioprine, campath 1H, FTY720 or an analogue thereof, an anti IL-8 antibody, mycophenolic acid or a salt thereof, e.g. Mycophenolate Mofetil (CELLCEPT®), OKT3 (ORTHOCLONE OKT3®), ATGAM®, THYMOGLOBULIN®, Brequinar Sodium, OKT4, T10B9.A-3A, 33B3.1, 15-deoxyspergualin, tresperimus, Leflunomide ARAVA®, CTLA1-Ig, anti-CD25, anti-IL2R, Basiliximab (SIMULECT®), Daclizumab (ZENAPAX®), mizorbine, ISAtx-247, CTLA4Ig (Abatacept), LEA29Y, LFA3Ig, hu5C8, etanercept (sold as Enbrel® by Immunex), adalimumab (Humira®), infliximab (Remicade®), an anti-LFA-1 antibody, natalizumab (Antegren®), UO126, B7RP-1-fc, hul 124, BTI-322, allotrap-HLA-B270, Enlimomab, ABX-CBL, antithymocyte immunoglobulin, Medi-500, Medi-507, Alefacept and efalizumab.

The combinations of the invention are appropriate for prevention of an allergic response as well as treating a pre-existing allergic condition.

The term “treatment” as used herein includes alleviation of one or more symptoms of the disorder, diminishment of the extent of the disorder, stabilization of the disorder, delay or slowing of disorder progression, amelioration or palliation of the disorder, and partial or total remission.

The terms “warm-blooded animal” or “mammal” include a human being.

The term “prevention” means prophylactic administration of the combination to healthy patients to prevent the outbreak of the diseases and conditions mentioned herein. Moreover, the term “prevention” means prophylactic administration of such combination to patients being in a pre-stage of the allergic disease to be treated.

The term “delay of progression” used herein means administration of the combination to patients being in a pre-stage of the allergic disease to be treated in which patients a pre-form of the corresponding disease is diagnosed.

The term “allergic disease” is to be understood according to its meaning in the art of medicine. In particular, allergic disease within the meaning of the invention includes a disease that is characterized by an allergic and/or atopic immunological reaction to an antigen, which results in allergic and/or atopic symptoms in the patient suffering from allergic disease. The term “allergic disease” in particular includes a disease which is characterized by elevated circulating IgE levels. An allergic disease often is characterized by the generation of antigen-specific IgE and the resultant effects of the IgE antibodies. As is well-known in the art, IgE binds to IgE receptors on mast cells and basophils. Upon later exposure to the antigen recognized by the IgE, the antigen cross-links the IgE on the mast cells and basophils causing degranulation of these cells.

Preferred examples of allergic disease are allergic asthma, seasonal allergic rhinitis, perennial allergic rhinitis, atopic dermatitis, chronic rhinosinusitis, COPD, food allergy such as e.g. peanut allergy.

Thus, in a preferred embodiment, the COMBINATION OF THE INVENTION is used to treat allergic asthma, seasonal allergic rhinitis, perennial allergic rhinitis, atopic dermatitis, chronic rhinosinusitis, COPD, food allergy such as e.g. peanut allergy.

In another preferred embodiment, the COMBINATION OF THE INVENTION is used to treat a combination of allergic diseases, e.g. allergic asthma with seasonal allergic rhinitis or perennial allergic rhinitis, or with atopic dermatitis, or with chronic rhinosinusitis, or with COPD or with food allergy such as e.g. peanut allergy. Other combinations include, but are not limited to, a combination of seasonal allergic rhinitis with perennial allergic rhinitis; seasonal allergic rhinitis or perennial allergic rhinitis with one of the following: atopic dermatitis, chronic rhinosinusitis, COPD, food allergy such as e.g. peanut allergy; atopic dermatitis with chronic rhinosinusitis, or with COPD or with food allergy such as e.g. peanut allergy; chronic rhinosinusitis with COPD, food allergy such as e.g. peanut allergy; COPD with food allergy such as e.g. peanut allergy.

In yet another preferred embodiment, COMBINATION OF THE INVENTION is used to treat or prevent rejection of transplanted organs, prevention of acute renal allograft rejection.

Allergic asthma as a clinical disorder that is characterized by airway inflammation; airway obstruction, which is reversible; and increased sensitivity, referred to as hyperreactivity. Obstruction to airflow is measured by a decrement in forced expired volume in one second (FEV I) which is obtained by comparison to baseline spirometry. Hyperreactivity of the airways is recognized by decreases in FEVI in response to very low levels of histamine or methacholine. Hyperreactivity may be exacerbated by exposure of the airways to allergen. Allergy testing can be helpful in identifying allergens in patients with persistent asthma. Common allergens include pet dander, dust mites, cockroach allergens, molds, and pollens. Common respiratory irritants include tobacco smoke, pollution, and fumes from burning wood or gas.

Allergic rhinitis is a clinical disorder characterized by nasal congestion, rhinorrhea, sneezing, and itching. Severity of these symptoms can vary from year to year, with occasional spontaneous remissions. Therefore, allergic rhinitis is classified by whether symptoms occur during certain seasons (SAR or seasonal allergic rhinitis) or year-round (PAR or perennial allergic rhinitis). The seasonal variety is usually caused by pollens from plants that depend on the wind for cross-pollination, such as grasses, trees, weeds, and mold spores. Serious complications, such as nasal polyps, recurrent sinusitis, recurrent ear infections, and hearing loss, can occur if allergic rhinitis is not treated or is undertreated. Psychosocial effects can include frequent absences from work or school, poor performance, poor appetite, malaise, and chronic fatigue.

Atopic dermatitis is a skin disorder involving hypersensitivity reaction within the skin characterized by inflammation, itching, and scaling. Atopic dermatitis can occur in an infantile or adult form. There is often a family history of asthma, hay fever, eczema, psoriasis, or other allergic diseases or allergy-related disorders. In adults, it is generally a chronic condition. Neurodermatitis is also a form of atopic dermatitis. It is characterized by a self-perpetuating scratch-itch cycle. Although symptoms increase in times of stress, physiological changes in the nerve fibers are also present. A hypersensitivity reaction occurs in the skin, causing chronic inflammation.

Food allergy is an allergic reaction that occurs when the immune system responds defensively to a specific food protein when ingested. In adults, the most common foods to cause allergic reactions include: shellfish such as shrimp, crayfish, lobster, and crab; peanuts, a legume that is one of the chief foods to cause severe anaphylaxis, a sudden drop in blood pressure that can be fatal if not treated quickly; tree nuts such as walnuts; fish; and eggs. In children, the pattern is somewhat different. The most common food allergens that cause problems in children are eggs, milk, and peanuts. Adults usually do not lose their allergies, but children can sometimes outgrow them. Children are more likely to outgrow allergies to milk or soy than allergies to peanuts, fish, or shrimp

Chronic Obstructive Pulmonary Disease (COPD) is a general term for several lung diseases that includes chronic bronchitis, emphysema and chronic asthma. COPD adds to the work of the heart, since the amount of oxygen that goes to the blood may be reduced. The two primary causes of COPD are cigarette smoking and alpha 1 antitrypsin deficiency. Air pollution and occupational dusts may also contribute to COPD, especially if the person exposed to these substances is a smoker.

Chronic rhinosinusitis is a group of disorders characterized by inflammation of the mucosa of the nose and paranasal sinuses lasting for at least 12 consecutive weeks. Factors associated with chronic rhinosinusitis include systemic (i.e., allergic, immunodeficiency, genetic/congenital, mucociliary dysfunction, endocrine, neuromechanism), local (i.e., anatomic, neoplastic, acquired mucociliary dysfunction), and environmental (i.e., microorganisms [viral, bacterial, fungal], noxious chemicals, pollutants, smoke, medications, trauma, surgery).

The nature of allergic disease and related diseases or conditions is multifactorial. Under certain circumstances, drugs with different mechanisms of action may be combined. However, just considering any combination of drugs having different mode of action but acting in the similar field does not necessarily lead to combinations with advantageous effects.

All the more surprising is the finding that the combined administration of a COMBINATION OF THE INVENTION, results in a beneficial, especially a synergistic, therapeutic effect and/or in additional benefits resulting from combined treatment such as a surprising prolongation of efficacy, a broader variety of therapeutic treatment and surprising beneficial effects on diseases and conditions associated with allergic asthma or seasonal allergic rhinitis compared to a monotherapy applying only one of the pharmaceutically active ingredients used in the COMBINATION OF THE INVENTION.

Accordingly, the invention provides a pharmaceutical composition which comprises an anti-IgE antibody such as, for example, Omalizumab and at least one immunosuppressive agent, or the pharmaceutically acceptable salts of such compounds where possible.

Preferably, the immunosuppressive agent is an antibody, in particular an anti-CD25 antibody. The antibodies are particularly suitable to be administered in a fixed combination for parenteral, and, in particular, subcutaneous administration.

Treatment with Omalizumab, respectively, can commence prior to, subsequent to or concurrent with commencement of treatment with the immunosuppressive agent of the invention.

It will be understood that any statistically significant attenuation in the disease symptoms of allergic disease, such as allergic asthma or seasonal allergic rhinitis pursuant to the treatment of the present invention is within the scope of the invention.

In practical use, the immunosuppressive agent can be combined as the active ingredients in intimate admixture with a pharmaceutical carrier according to conventional pharmaceutical compounding techniques. The carrier may take a wide variety of forms depending on the form of preparation desired for administration, e.g. subcutaneous administration, oral or parenteral (including intravenous). In preparing the compositions for oral or subcutaneous dosage form, any of the usual pharmaceutical media may be employed or carriers, diluents, granulating agents, lubricants, binders, disintegrating agents and the like. In the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. The preferred dosage form is either as a liquid, or a reconstituted solution.

Preferred routes of administration are subcutaneous or intravenous. Also preferred are intra-nasal or inhaled delivery of the COMBINATION OF THE INVENTION.

In a particularly preferred embodiment, COMBINATION OF THE INVENTION is formulated as described in WO97/04801.

A further aspect of the present invention is the use of a pharmaceutical composition comprising the COMBINATION OF THE INVENTION for the preparation of a medicament for the prevention, delay of progression or treatment of allergic disease, in particular of allergic asthma, seasonal allergic rhinitis, perennial allergic rhinitis, atopic dermatitis, chronic rhinosinusitis, COPD, food allergy such as e.g. peanut allergy or a disease or condition associated with allergic disease.

There is further provided a method of prevention, delay of progression or treatment of and a pharmaceutical composition for the prevention, delay of progression or treatment of allergic disease. The treatment involves administering to a patient in need of such treatment a pharmaceutical composition comprising a pharmaceutical carrier and a therapeutically effective amount of the COMBINATION OF THE INVENTION.

A further aspect of the present invention is a method of treatment of a warm-blooded animal, especially a human, having allergic disease, in particular allergic asthma, seasonal allergic rhinitis, perennial allergic rhinitis, atopic dermatitis, chronic rhinosinusitis, COPD, food allergy such as e.g. peanut allergy or a disease or condition associated with allergic disease, comprising administering to the animal a COMBINATION OF THE INVENTION in an amount which is jointly therapeutically effective against allergic disease in which the active ingredients can also be present in the form of their pharmaceutically acceptable salts simultaneously or sequentially in any order, separately or in a fixed combination.

In a preferred embodiment of the above aspects, there is provided a method of prevention, delay of progression or treatment of a warm-blooded animal, especially a human, having a combination of allergic disease, e.g. allergic asthma with seasonal allergic rhinitis or perennial allergic rhinitis, or with atopic dermatitis, or with chronic rhinosinusitis, or with COPD or with food allergy such as e.g. peanut allergy. Other combinations include, but are not limited to, a combination of seasonal allergic rhinitis with perennial allergic rhinitis; seasonal allergic rhinitis or perennial allergic rhinitis with one of the following: atopic dermatitis, chronic rhinosinusitis, COPD, food allergy such as e.g. peanut allergy; atopic dermatitis with chronic rhinosinusitis, or with COPD or with food allergy such as e.g. peanut allergy; chronic rhinosinusitis with COPD, food allergy such as e.g. peanut allergy; COPD with food allergy such as e.g. peanut allergy.

In yet another preferred embodiment of the above aspects, there is provided a method of preventing the rejection of transplanted organs, e.g. an acute renal allograft rejection.

The invention relates in particular to a kit or commercial package comprising jointly therapeutically effective amounts of COMBINATION OF THE INVENTION together with instructions for use thereof in the treatment of allergic disease, in allergic asthma, seasonal allergic rhinitis, perennial allergic rhinitis, atopic dermatitis, chronic rhinosinusitis, COPD, food allergy such as e.g. peanut allergy or a disease or condition associated with allergic disease.

The therapeutically effective dosage of each of the active ingredients employed in the combination therapy may vary depending on the particular compound or pharmaceutical composition employed, the mode of administration, the condition being treated, the severity of the condition being treated, the species of the warm-blooded animal, body weight, sex, diet and age. Thus, the dosage regimen utilizing the compounds of the present invention is selected in accordance with a variety of factors including the route of administration and the renal and hepatic function of the patient. A physician, clinician or veterinarian of ordinary skill can readily determine and prescribe the effective amount of the drug required to prevent, counter or arrest the progress of the condition. Optimal precision in achieving concentration of drug within the range that yields efficacy without toxicity requires a regimen based on the kinetics of the drug's availability to target sites. This involves a consideration of the distribution, equilibrium, and elimination of a drug. Hence, the dosage regimen, i.e. dose level and frequency of dosage, of any of the individual components of the COMBINATION OF THE INVENTION as described hereinafter may be adjusted to provide the optimal therapeutic response.

By “co-administration” is meant administration of the components of the compositions of the invention together or at substantially the same time, e.g. within fifteen minutes or less, either in the same vehicle or in separate vehicles, so that upon administration, for example, both compounds are present simultaneously in the gastrointestinal tract. The compounds may be administered as a fixed combination or may be administered in separate dosage forms.

It will be appreciated that the unit content of active ingredient or ingredients contained in an individual dose of each dosage form need not in itself constitute an effective amount since the necessary effective amount can be reached by administration of a plurality of dosage units.

The invention will be more fully understood by reference to the following examples. They should not, however, be construed as limiting the scope of the invention.

### EXAMPLE 1

It can be shown by established test models and especially those test models described herein that the COMBINATION OF THE INVENTION results in a more effective prevention or, preferably, treatment of allergic disease, and in particular allergic asthma, seasonal allergic rhinitis, perennial allergic rhinitis and atopic dermatitis and diseases and conditions associated with allergic disease.

The person skilled in the pertinent art is fully enabled to select a relevant in vitro or animal test model to assess the hereinbefore and hereinafter indicated therapeutic indications and beneficial effects. Often the person skilled in the pertinent art will conduct a relevant clinical study to assess the hereinbefore and hereinafter indicated therapeutic indications and beneficial effects. For example, the anti-IgE antibody of the composition or combination to be tested may not cross-react with IgE from small mammals, other than primates and man.

### Clinical Studies

Clinical studies prove, e.g., the synergism of the COMBINATION OF THE INVENTION. The beneficial effects on allergic disease and conditions associated with allergic disease as defined in this application can be determined directly through the results of these studies or by changes in the study designs which are known as such to a person skilled in the art.

The studies are, in particular, suitable to assess the effects of monotherapy with an anti-IgE antibody such as, for example, Omalizumab, and the other active ingredients mentioned herein in comparison to a COMBINATION OF THE INVENTION on for example, exacertion rates, symptom control, concomitant medication in use or other relevant functional parameters of allergic disease, such as for example the lung function. Measurement of free IgE may also serve as a marker of therapeutical efficacy. The length of the respective study depends on the combination to be tested, in many cases a duration of at least 16 weeks is likely to be needed.

Example for a Clinical Double-Blind, Randomized, Parallel-Group Studies in Subjects with Allergic Asthma to Assess Efficacy of an an Anti-IgE Antibody Administered Together with a Combination Partner

Subjects with a diagnosis of allergic asthma are chosen for these trials. The effects on the reduction of rescue medication intake (such as antihistamines and corticosteroids) and/or reduction of clinical symptoms are determined in this studies with the control achieved on placebo.

Efficacy parameter scores are mean and median daily symptom scores which are calculated based on the patient's diary assessment of clinical symptoms. Symptoms are categorized into 7 domains (stuffy nose, runny nose, itchy nose, sneezing and itchy eyes, watery eyes, red eyes). Each category can score 0-3 (none-mild-moderate-severe). Daily rescue medication scores are given: 0 for no medication; 1 for topical antihistamines; 2 for systemic antihistamines, 3 for oral or topical corticosteroids. Only maximal score per day is assessed.

The primary outcome variable is the symptom load (mean daily symptom score plus mean daily rescue medication score). Secondary clinical efficacy variables measured are: symptom score (mean of the daily symptom score), rescue medication score (mean of the daily rescue medication score during entire pollen season), proportion of days with rescue and/or concomitant medication use, investigator's global evaluation of treatment tolerability.

Safety assessments include monitoring and recording of all adverse events and serious adverse events, hematological, serum chemistry and urinary laboratory evaluations.

Before starting with the double-blind treatment for 16-24 weeks, the subjects are administered for 4 weeks an anti-IgE antibody such as, for example, Omalizumab, matching placebos and a placebo matching the combination partner (period I). The subjects are then separated into four treatment groups for the 16-24 weeks double-blind study (period II) as depicted in Table 1. Approximately 50 to 250 subjects are randomized per treatment group. The total study duration including the run-in period for each subject can be, e.g 20-28 weeks. Statistical analysis can be carried out by methods known in the art.

TABLE 1
Treatment groups for the double-blind study

1.) anti-IgE antibody* + combination partner placebo
2.) anti-IgE antibody placebo* + combination partner
3.) anti-IgE antibody* + combination partner
4.) anti-IgE antibody placebo* + combination partner placebo
*administered subcutaneously

### In One Clinical Study Where the Anti-IgE Antibody is Omalizumab, the Following Administration and Dosage Scheme is Chosen:

Omalizumab is supplied as a sterile, freeze dried preparation that can be reconstituted to a final Omalizumab concentration of 125 mg/ml. Each 10 ml vial contains 208 mg rhuMAb-E25. Omalizumab must be stored refrigerated at (2°-8° C.) until time of administration to the subject, do not freeze. Each vial is reconstituted with 1.3 ml of Sterile Water for Injection (SWI), and the contents are gently swirled for 30 seconds, then left for up to 5 minutes to solubilize. 1.2 ml is then drawn up to deliver 150 mg of rhuMAb-E25. The formulation does not contain a preservative and is to be used for single-dose administration only.

After reconstitution, patients randomized to Omalizumab receive blinded test drug dependent on baseline IgE levels. The corresponding placebo group receive placebo dependent on IgE levels.

Omalizumab is administered using a disposable 25 gauge needle and a disposable plastic tuberculin-type syringe. The injections are administered in the deltoid region on the right arm. Alternately, the injections can be administered in the right thigh if medically significant reasons preclude administration in the deltoid region. The injections are administered subcutaneously.

The dose of Omalizumab which is based on baseline free serum IgE levels, is designed to suppresses free serum IgE to levels below 25 ng/ml. For example, in patients with asthma, Omalizumab 150-375 mg may be administered subcutaneously every 2 or 4 weeks (Tables 2 and 3).

TABLE 2
Omalizumab Dosing Schedule/Number of injections per dose (mg)

| Dose | Number | Injection volume |
|---|---|---|
| (mg) | of injections | (mL) |
| 150 | 1 | 1.2 |
| 225 | 2 | 1.8 (1.2 + 0.6) |
| 300 | 2 | 2.4 (1.2 + 1.2) |
| 375 | 3 | 3.0 (1.2 + 1.2 + 0.6) |

The dose of Omalizumab is based on both the patient's body weight and their total serum IgE level measured before treatment, according to the scheme shown in Table 3.

TABLE 3
Omalizumab doses, SQ Administration

| Baseline | Milligrams (mg) Per Dose | | | | | | |
|---|---|---|---|---|---|---|---|
| IgE | Body weight (kg) | | | | | | Frequency |
| (IU/mL) | 20-30 | >30-40 | >40-50 | >50-60 | >60-70 | >70-90 | of Dosing |
| >30-100 | 150 | 150 | 150 | 150 | 150 | 150 | Q4wk |
| >100-200 | 150 | 150 | 300 | 300 | 300 | 300 | |
| >200-300 | 150 | 300 | 300 | 300 | 225 | 225 | Q2wk |
| >300-400 | 300 | 300 | 225 | 225 | 225 | 300 | |
| >400-500 | 300 | 225 | 225 | 300 | 300 | 375 | |
| >500-600 | 300 | 225 | 300 | 300 | 375 | | Not Dosed |
| >600-700 | 225 | 225 | 300 | 375 | | | |
| >700-800 | 225 | 300 | 375 | | | | |
| >800-900 | 225 | 300 | 375 | | | | |
| >900-1000 | 300 | 375 | | | | | |
| >1000-1100 | 300 | 375 | | | | | |
| >1100-1200 | 300 | | | | | | |
| >1200-1300 | 375 | | | | | | |

The 2-weekly schedule (Q2wk) should be adopted if the dose will present too large a volume of injection for administration at one visit.

Various parameters of the study described above can be modified, e.g. in order to optimize the dosage for special diseases or indications mentioned herein, to cope with tolerability problems during the study or to obtain similar or identical results with less efforts. For example, a different subject population can be involved in such a clinical trial, the term of the placebo run-in period (period I) can be changed, i.e. it can be extended, shortened or deleted; the visit schedule can be extended; the visit instructions can be changed; or one or more of the parameters to be determined during the study mentioned above can be deleted or the determination of additional parameters (see below) can be added.

Additional parameters can be determined in the course of the study, e.g. by additional tests. For example, a peak flow meter, a simple device to measure lung volume, can be used at home daily to check on lung functions. Peak flow values of 50-80% of an individual's personal best indicate a moderate asthma exacerbation, while values below 50% indicate a severe exacerbation.

### Results

The combined administration of the COMBINATION OF THE INVENTION results in a beneficial, especially a synergistic, therapeutic effect, especially on allergic disease, and/or in additional benefits, or an improved safety profile, compared to a monotherapy applying only one of the active ingredients used in the COMBINATION OF THE INVENTION. Further benefits are, e.g., that lower doses of the individual drugs to be combined according to the present invention can be used to reduce the dosage, for example, that the dosages need not only often be smaller but are also applied less frequently, or can be used in order to diminish the incidence of side effects.

Furthermore, in a number of combinations as disclosed herein the side-effects observed with one of the active ingredients surprisingly do not accumulate on application of the COMBINATION OF THE INVENTION.

Additionally, the beneficial therapeutic effects, additional benefits and also the surprising beneficial effects are observed especially in patients poorly controlled by monotherapy with one of the components of the COMBINATION OF THE INVENTION.

1. A pharmaceutical composition comprising as active ingredients an Anti-IgE Antibody and at least one immunosuppressive agent in which the active ingredients are present in each case in free form or in the form of a pharmaceutically acceptable salt and optionally at least one pharmaceutically acceptable carrier; for simultaneous, separate or sequential use.

2. The composition according to claim 1 wherein the Anti-IgE Antibody and the at least one further immunosuppressive agent is comprised in a single pharmaceutical formulation.

3. The composition according to claim 1 wherein the immunosuppressive agent is selected from the group consisting of a Cyclosporin A (NEORAL®), ISAtx-247, FK778, ABT-281 or ASM981, azathioprine, campath 1H, FTY720 or an analogue thereof, an anti IL-8 antibody, mycophenolic acid or a salt thereof, e.g. Mycophenolate Mofetil (CELLCEPT®), OKT3 (ORTHOCLONE OKT3®), ATGAM®, THYMOGLOBULIN®, Brequinar Sodium, OKT4, T10B9.A-3A, 33B3.1, 15-deoxyspergualin, tresperimus, Leflunomide ARAVA®, CTLA1-Ig, anti-CD25, anti-IL2R, Basiliximab (SIMULECT®), Daclizumab (ZENAPAX®), mizorbine, ISAtx-247, CTLA4Ig (Abatacept), LEA29Y, LFA3Ig, hu5C8, etanercept (sold as Enbrel® by Immunex), adalimumab (Humira®), infliximab (Remicade®), an anti-LFA-1 antibody, natalizumab (Antegren®), UO126, B7RP-1-fc, hul 124, BTI-322, allotrap-HLA-B270, Enlimomab, ABX-CBL, antithymocyte immunoglobulin, Medi-500, Medi-507, Alefacept and efalizumab.

4. A composition according to claims 3 wherein immunosuppressive agent is selected from the group consisting of an anti-IL2R antibody, an anti-CD25 antibody, basiliximab, daclizumab, an anti IL-8 antibody, adalimumab, infliximab, hu5C8, OKT3, OKT4, anti-TACac, T10B9.A-3A, 33B3.1, hul 124, BTI-322, allotrap-HLA-B270, Enlimomab, ABX-CBL, antithymocyte immunoglobulin, ISAtx247, Medi-500, Medi-507, Alefacept and efalizumab.

5. A composition according to claim 1 wherein immunosuppressive agent inhibits T-cell activation.

6. A composition according to claim 5 wherein the immunosuppressive agent inhibits T-cell activation by at least 10%.

7. (canceled)

8. The composition according to claim 1 wherein the Anti-IgE Antibody is Omalizumab.

9. The composition according to claim 8, characterized in that Omalizumab is administered in a concentration dependent on the baseline IgE level of the patient to be treated.

10. The composition according to claim 1, wherein the Anti-IgE Antibody and the immunosuppressive agent are present in respective amounts which are jointly therapeutically effective against allergic disease.

11. The composition according to claim 10 wherein the Anti-IgE Antibody and the immunosuppressive agent are present in respective amounts which are jointly therapeutically effective against asthma.

12-16. (canceled)

17. A kit comprising a composition according to claim 1 together with instructions for simultaneous, separate or sequential use thereof in the treatment of allergic disease.

18. A method of treating an allergic disease with a composition according to claim 1.

19. A method of treating an allergic disease according to claim 18 wherein said allergic disease is selected from the group consisting of allergic asthma, seasonal allergic rhinitis, perennial allergic rhinitis, atopic dermatitis, chronic rhinosinusitis, COPD, food allergy and diseases and conditions associated with allergic disease or combinations thereof.

20. A method of treating the rejection of transplanted organs or acute renal allograft rejection with a composition according to claim 1.
